(19) United States
(12) Patent Application Publication
(10) Pub. No.: US 2012/0088280 A1
(43) Pub. Date: Apr. 12, 2012

(54) GENE ENCODING POLYMER SYNTHASE AND A PROCESS FOR PRODUCING POLYMER

(76) Inventors: K.Sudesh Kumar C.Kanapathi Pillai, Pulau Pinang (MY); Mohammed Razip Bin Samian, Pulau Pinang (MY); Amirul Al-Ashraf Balakrishnan Bin Abdullah, Pulau Pinang (MY); Kesivan Bhubalan, Pulau Pinang (MY)

(21) Appl. No.: 13/377,221

(22) PCT Filed: May 5, 2010
(86) PCT No.: PCT/MY2010/000071

§ 371 Date: Dec. 9, 2011

(30) Foreign Application Priority Data
Jun. 12, 2009 (MY) PI 2009 2412

(51) Int. Cl. C12P 7/62; C07H 21/04; C12N 1/19; C12N 15/63; C12N 5/10; C12N 1/21

(52) U.S. Cl. 435/135; 435/325; 435/358; 435/365; 435/252.3; 435/252.31; 435/252.34; 435/254.21; 435/254.22; 435/320.1; 536/23.2

(57) ABSTRACT

An isolated polynucleotide encoding for a polypeptide comprising an amino acid sequence set forth in SEQ ID NO: 1 with polymer synthase activity.

### FIELD OF INVENTION

The present invention relates to a polymer synthase and a gene encoding for this enzyme. In more particular, the present invention provides a functional gene encoding for an enzyme of polymer synthase, a recombinant vector containing the gene, a transformant transformed by the vector, and a process for producing polymer synthase which relates to the synthesis of plastic-like polymer by use of the transformant.

### BACKGROUND OF THE INVENTION

Polyhydroxyalkanoates (PHAs) are microbial storage polymers with properties that closely resemble the properties of main commodity plastics. Most PHAs are thermoplastics and can be thermally processed like the petrochemical-derived synthetic plastics with an added advantage of biodegradability. PHAs are also renewable by nature as they can be produced from renewable resources such as sugars, plant oils and carbon dioxide. Poly(3-hydroxybutyrate) [P(3HB)] is the first type of PHA to be identified and is the most common PHA found in nature.

The properties of PHA can be tailored to suit various applications by controlling the incorporation and/or composition of secondary monomers. Polymer synthesizing microorganisms can be divided into 2 groups, which are those synthesizing polymers with C3 to C5 monomer units and those synthesizing polymers with C6 to C14 monomer units. These respective microorganisms possess substrate specific polymer synthase. Polymer consisting of at least C6 monomer units is soft polymeric materials with elastomeric properties.

Ever growing interest in PHA has resulted in isolation of new bacterial strains for improved and novel polymer production. Production of PHA by both Gram negative and Gram positive microorganisms have been investigated and well documented. The genes involved in PHA biosynthesis including its key enzyme, the PHA synthase, from these microorganisms has been identified and characterized.

There are some patented technologies over the prior arts relating to a polymer synthase and the gene coding therefor. U.S. Pat. No. 6,812,013 relates to a PHA synthase useful in a process for preparing a PHA, a gene encoding this enzyme, a recombinant vector comprising the gene, a transformant transformed by the vector, a process for producing a PHA synthase utilizing the transformant and a process for preparing a PHA utilizing the transformant. This invention is characterized by a transformant obtained by introducing a PHA synthase gene from Pseudomonas putida into a host microorganism which is cultured to produce a PHA synthase or PHA.

Another U.S. Patent No. US2004146998 also relates to a transformant and process for producing polymer by using the same. This invention discloses a gene encoding for a copolymer-synthesizing enzyme, a microorganism which utilizes the gene for the fermentative synthesis of a polymer and a method of producing a polymer with the aid of the microorganism. This invention focuses on the construction of the transformant which comprises a polyester synthesis-associated enzyme gene, a promoter and a terminator and has been introduced into yeast.

An improved transformant and process for producing polymer using the same are disclosed in U.S. Patent No. EP1626087. This invention provides a gene expression cassette which comprises a gene coding for an Aeromonas caviae-derived PHA synthase. Yeast is also used as a host and a mutation has been introduced in the promoter and terminator so as to allow the gene cassette to be functioning in the yeast.

Some of the patented technologies disclose a combination between polymer synthase encoding gene and other genes. U.S. Patent No. US2008233620 relates to a transformant and a process for producing a gene expression product in yeast. The transformant is obtained by introducing a plurality of enzyme genes involved in PHA synthesis such as a combination of PHA synthase and an acetoacetyl CoA reductase gene. In another U.S. Patent No. US2003146703, a recombinant microorganism expressing both PHA synthase and intracellular PHA depolymerase is disclosed. This invention allows the simultaneous synthesis and degradation of PHA.

Most of the patented technologies relate to a transformant and a process for producing polymer or PHA using the transformant disclosed. However, these patented technologies involve PHA synthase genes which are derived from a different region of the genome of a different species of organism. Thus far, there is also no patented technology disclosing the incorporation of C3 to C7 monomer units in the synthesis of a polymer synthase. It is therefore desirable for the present invention to provide an improved DNA fragment of the polymer synthase gene to produce a recombinant vector and a transformant which can be useful in providing polymer synthase with increased level of activity.

### SUMMARY OF INVENTION

The primary object of the present invention is to provide a polymer synthase gene which is derived from bacterial species, and a synthesis of polymer synthase encoded by the gene with the incorporation of useful monomer units.

Another object of the present invention is to provide a new DNA fragment of the polymer synthase to be ligated into a suitable vector to produce a recombinant vector, and hence to provide a transformant containing the polymer synthase.

Still another object of the present invention is to provide a polymer synthase with increased level of activity.

Yet another object of the present invention is to develop a more efficient method for producing polymers using the transformant containing the polymer synthase.

At least one of the preceding objects is met, in whole or in part, by the present invention, in which one of the embodiments of the present invention describes an isolated polynucleotide encoding for a polypeptide comprising an amino acid sequence set forth in SEQ ID NO: 1 with polymer synthase activity.

Another embodiment of the present invention is an isolated polynucleotide encoding for a polypeptide comprising an amino acid sequence set forth in SEQ ID NO: 1, wherein one or more amino acids is replaced, deleted, replaced or added, the polypeptide having polymer synthase activity.

According to the preferred embodiment of the present invention, the isolated polynucleotide comprises a nucleotide sequence set forth in SEQ ID NO: 2 or the complementary sequence thereof.

Still another preferred embodiment of the present invention is an isolated polynucleotide comprising a nucleotide sequence set forth in SEQ ID NO: 2, wherein T is replaced by U; or the complementary sequence thereof.

Yet another embodiment of the present invention is a recombinant vector comprising an isolated polynucleotide, wherein the isolated polynucleotide is encoding for a polypeptide comprising an amino acid sequence set forth in SEQ ID NO: 1 with polymer synthase activity; or a polypeptide comprising an amino acid sequence set forth in SEQ ID NO: 1, wherein one or more amino acids is replaced, deleted, replaced or added, the polypeptide having polymer synthase activity. Preferably, the recombinant vector is a plasmid.

In a further embodiment of the present invention, a transformant transformed by the vector as set forth in the preceding embodiments is disclosed.

Another further embodiment of the present invention is a process for producing polymer comprising: culturing a transformant comprising an isolated polynucleotide as set forth in any of the preceding embodiments in a medium containing polymerizable materials; and recovering the polymer from the cultured medium. Preferably, the polymer is PHA.

One skilled in the art will readily appreciate that the present invention is well adapted to carry out the objects and obtain the ends and advantages mentioned, as well as those inherent therein. The embodiments described herein are not intended as limitations on the scope of the invention.

### BRIEF DESCRIPTION OF THE DRAWINGS

For the purpose of facilitating an understanding of the invention, there is illustrated in the accompanying drawing the preferred embodiments from an inspection of which when considered in connection with the following description, the invention, its construction and operation and many of its advantages would be readily understood and appreciated.

FIG. 1 is the amino acid sequence of the polypeptide of polymer synthase as described in one of the preferred embodiments of the present invention.

FIG. 2 is the nucleotide sequence of the polynucleotide encoding the polymer synthase as described in one of the preferred embodiments of the present invention.

FIG. 3 is the nucleotide sequences of the amplification nucleotides used for the PCR amplification of the polymer synthase as described in one of the preferred embodiments of the present invention.

FIG. 4 is the H-NMR spectrum of P(3-hydroxybutyrate-co-3-hydroxyvalerate-co-3-hydroxyhexanoate), one of the example of the copolymer synthesized by the transformant of the as described in one of the preferred embodiments of the present invention.

### DETAILED DESCRIPTION OF THE INVENTION

The present invention relates to a polymer synthase and a gene encoding for this enzyme. In more particular, the present invention provides a functional gene encoding for an enzyme of polymer synthase, a recombinant vector containing the gene, a transformant transformed by the vector, and a process for producing polymer synthase which relates to the synthesis of plastic-like polymer by use of the transformant.

Hereinafter, the invention shall be described according to the preferred embodiments of the present invention and by referring to the accompanying description and drawings. However, it is to be understood that limiting the description to the preferred embodiments of the invention and to the drawings is merely to facilitate discussion of the present invention and it is envisioned that those skilled in the art may devise various modifications without departing from the scope of the appended claim.

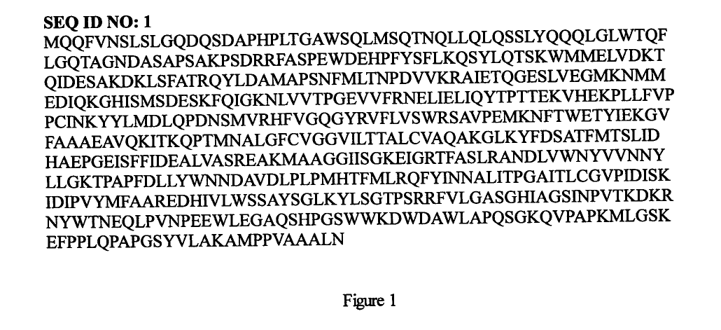

The present invention discloses an isolated polynucleotide encoding for a polypeptide comprising an amino acid sequence set forth in SEQ ID NO: 1 with polymer synthase activity. SEQ ID NO: 1 is illustrated in FIG. 1.

According to the preferred embodiment of the present invention, the isolated polynucleotide is a polymer synthase gene. Besides, this polymer synthase gene can encode a polypeptide containing the amino acid sequence of SEQ ID NO: 1, or a sequence where one or more amino acids are deleted from, replaced with or added to the amino acid sequence of SEQ ID NO: 1. Even if one or more amino acids in the sequence of SEQ ID NO: 1 may have undergone mutations such as deletion, replacement, or addition, the polynucleotide encoding for a polypeptide containing the amino acid sequence is contained in the gene of the present invention insofar as the polypeptide has polymer synthase activity. For example, polynucleotide encoding for the amino acid sequence of SEQ ID NO: 1 where methionine at the first position is deleted is also contained in the gene of the present invention. In other words, the gene of the present invention encompasses not only the nucleotide sequence coding for the amino acid sequence of SEQ ID NO: 2 but also its degenerated which except for degeneracy codons, code for the same polypeptide. The abovementioned mutations such as deletion, replacement or addition can be induced by known site-directed mutagenesis.

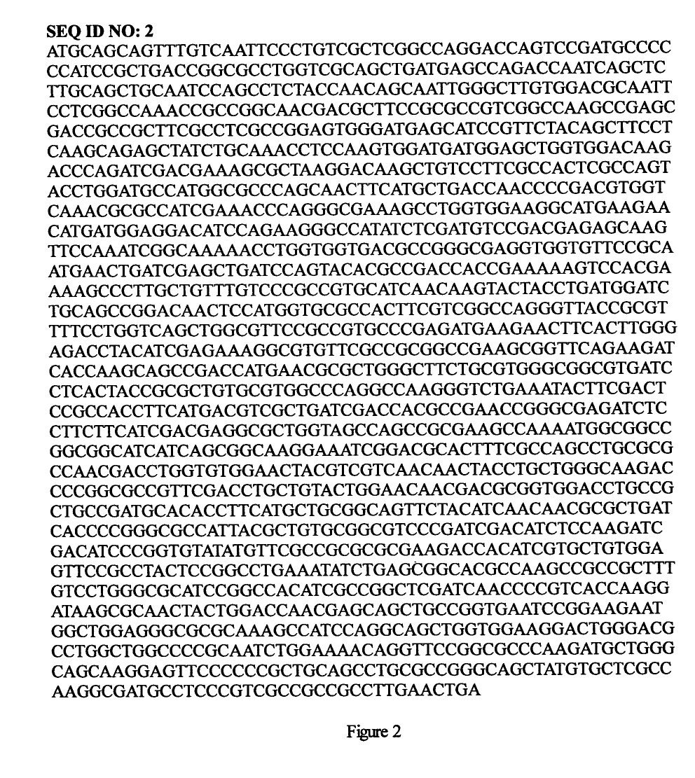

In a preferred embodiment of the present invention, an isolated polynucleotide comprising a nucleotide sequence set forth in SEQ ID NO: 2 or the complementary sequence thereof is disclosed. SEQ ID NO: 2 is shown in FIG. 2. Still another embodiment of the present invention is an isolated polynucleotide which comprises a nucleotide sequence set forth in SEQ ID NO: 2, wherein T is replaced by U; or the complementary sequence thereof. These polynucleotides are coding for a polypeptide with polymer synthase activity.

This polymer synthase gene is preferably cloned from a suitable microorganism. In accordance with the preferred embodiment of the present invention, the polymer synthase gene is separated from a microorganism belonging to the genus of Chromobacterium isolated from fresh water. The gene of the present invention can be obtained by chemical synthesis or the polymerase chain reaction (PCR) technique using genomic DNA as a template, or by hybridization using a DNA fragment having the nucleotide sequence as a probe.

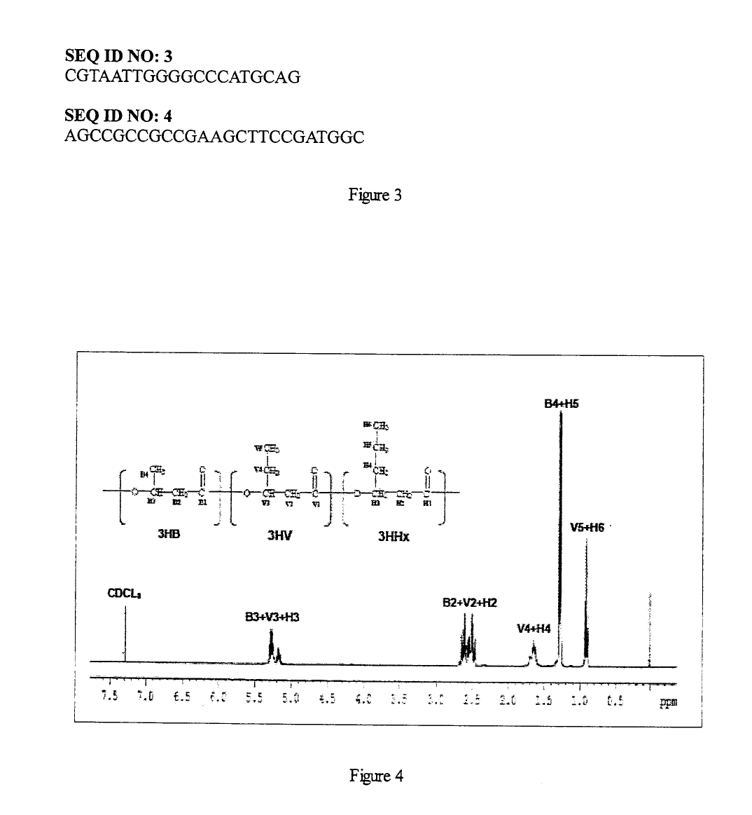

In accordance with the preferred embodiment of the present invention, PCR detection method is applied to obtain the DNA fragment of the polymer synthase gene using the genomic DNA from Chromobacterium sp. as template. Initially, the genomic DNA is isolated from the strain of Chromobacterium sp. It is known in the art that any suitable medium, for instance, a nutrient rich medium, can be used for the preparation of genomic DNA. To obtain a DNA fragment containing the polymer synthase gene derived from Chromobacterium sp., a probe is preferably prepared. Well-conserved regions of the polymer synthase gene are selected from the known amino acid sequence and nucleotide sequences coding for them can be estimated to design oligonucleotides. A primer pair of amplification nucleotides is designed to achieve this purpose. An example of the amplification nucleotides is shown in FIG. 3, in which SEQ ID NO: 3 is used as the forward primer and SEQ ID NO: 4 is used as the reverse primer.

The amplified DNA fragment can be digested with a suitable restriction enzyme, for example ApaI and SalI. The DNA fragment is then dephosphorylated by treatment with alkaline phosphatase. It is ligated into a vector previously cleaved with a restriction enzyme, which can be ApaI and SalI.

Yet another embodiment of the present invention is a recombinant vector comprising an isolated polynucleotide, wherein the isolated polynucleotide is encoding for a polypeptide comprising an amino acid sequence set forth in SEQ ID NO: 1 with polymer synthase activity; or a polypeptide comprising an amino acid sequence set forth in SEQ ID NO: 1, wherein one or more amino acids is replaced, deleted, replaced or added, the polypeptide having polymer synthase activity.

In accordance with the preferred embodiment of the present invention, plasmid or phage capable of autonomously replicating in host microorganism is used as the vector. The plasmid vector which can be applied includes pBR322, pUC18, and pBluescript II, whereas the phage vector which can be applied includes EMBL3, M13, lambda gt11. These vectors can be commercially obtained. Vectors capable of autonomously replicating in 2 or more host cells such as Escherichia coli or Bacillus brevis, as well as various shuttle vectors, can also be used. Such vectors are also cleaved with the restriction enzymes so that their fragment can be obtained.

Accordingly, conventional DNA ligase is used to ligate the resulting DNA fragment into the vector fragment. The DNA fragment and the vector fragment are annealed and then ligated to produce a recombinant vector.

In a further embodiment of the present invention, a transformant is obtained by introducing the recombinant vector of the present invention into a host compatible with the expression vector used in constructing said the recombinant vector. The present invention is not intended to limit the use of particular host as long as it is capable of expressing the target gene. Suitable examples that can be used are microorganisms belonging to the genus of Cupriavidus, Pseudomonas or Bacillus; or yeasts from the genus of Saccharomyces or Candida; or animal cells such as COS or CHO cell lines.

If bacteria such as microorganisms belonging to the genus Cupriavidus or Pseudomonas are used as the host, the recombinant DNA of the present invention is preferably constituted such that it contains a promoter, the DNA fragment of the present invention, and a transcription termination sequence. This is to ensure the occurrence of autonomous replication in the host. Preferably, the expression vector includes but not limited to pGEM-T and pBBR1MCS-2 derivatives. Likewise, the promoter can be of any type provided that it can be expressed in the host. Examples of promoters which are derived from E. coli or phage include trp promoter, lac promoter, pL promoter, pR promoter and T7 promoter.

To introduce the recombinant vector into a host microorganism, any known methods can be used. For example, if the host microorganism is E. coli, the calcium method and the electroporation method can be used. If phage DNA is used, the in vitro packaging method can be adopted.

Expression vectors such as Yep13 or YCp50 are employed if yeast is used as the host. Accordingly, the promoter can be gal 1 promoter or gal 10 promoter; and the method for introducing the recombinant DNA into yeast includes the electroporation method, the spheroplast method and the lithium acetate method. If animal cells are used as the host, expression vectors such as pcDNAI or pcDNAI/Amp are used. Accordingly, the method for introducing the recombinant DNA into animal cells can be the electroporation method or the potassium phosphate method.

The present invention also discloses a process for producing polymer comprising the steps of culturing a transformant comprising an isolated polynucleotide as set forth in any of the preceding embodiments in a medium containing polymerizable materials; and recovering the polymer from the cultured medium. The polymer can be formed and accumulated in the transformant.

A conventional method used for culturing the host is also used to culture the transformant of the present invention. The medium for the transformant prepared from a microorganism belonging to the genus Cupriavidus or Pseudomonas as the host include a medium containing a carbon source assimilable by the microorganism, in which a nitrogen source, inorganic salts or another organic nutrition source has been limited, for example a medium in which the nutrition source is in a range of 0.01% to 0.1% by weight of the medium.

The carbon source is necessary for growth of the microorganism, and it is simultaneously a starting material of polymer. The carbon source used can be derived from hydrocarbons such as glucose, fructose, sucrose or maltose. Further, fat- and oil-related substances having two or more carbon atoms can also be used as the carbon source. These fat- and oil-related substances include natural fats and oils, such as corn oil, soybean oil, safflower oil, sunflower oil, olive oil, coconut oil, palm oil, rape oil, fish oil, whale oil, porcine oil and cattle oil; aliphatic acids such as acetic acid, propionic acid, butanoic acid, pentanoic acid, hexoic acid, octanoic acid, decanoic acid, lauric acid, oleic acid, palmitic acid, linolenic acid, linolic acid and myristic acid as well as esters thereof; alcohols such as ethanol, propanol, butanol, pentanol, hexanol, octanol, lauryl alcohol, oleyl alcohol and palmityl alcohol as well as esters thereof. Meanwhile, the nitrogen source can be derived from ammonia, ammonium salts, peptone, meat extract, yeast extract or corn steep liquor. The inorganic matter includes monopotassium phosphate, dipotassium phosphate, magnesium phosphate, magnesium sulfate and sodium chloride.

The culture is preferably carried out under aerobic conditions with shaking at 30° C. to 34° C. for more than 24 hours, preferably 1 to 3 days, after expression is induced. During culture, antibiotics such as ampicillin, kanamycin, gentamycin, antipyrine or tetracycline can be added to the culture. Accordingly, the polymer can be accumulated in the microorganism, and the polymer can then be recovered.

To culture the microorganism transformed with the expression vector using an inducible promoter, its inducer, such as isopropyl-β-D-thiogalactopyranoside (IPTG) or indoleacrylic acid (IAA), can also be added to the medium. To culture the transformant from animal cells as the host, medium such as RPMI-1640 or DMEM supplemented with fetal bovine serum can be used. According to the preferred embodiment of the present invention, culture is carried out usually in the presence of 5% CO₂ at 30° C. to 37° C. for 14 to 28 days. During culture, antibiotics such as kanamycin or penicillin may be added to the medium.

In accordance with the preferred embodiment of the present invention, a polymer purification step can also be carried out. Preferably, the transformant is recovered from the culture by centrifugation, then washed with distilled water and hexane, and dried. Thereafter, the dried transformant is suspended in chloroform and heated to extract the polymer therefrom. The residues can are removed by filtration. Preferably, methanol is added to this chloroform solution to precipitate polymer. After the supernatant is removed by filtration or centrifugation, the precipitates are dried to give purified polymer. The resulting polymer is confirmed to be the desired one in a usual manner, for instance, by gas chromatography, nuclear magnetic resonance or others.

This polymer synthase can synthesize a copolymer (polymer) consisting of a monomer unit 3-hydroxyalkanoic acid represented by Formula I, wherein R represents a hydrogen atom or a C1 to C4 alkyl group.

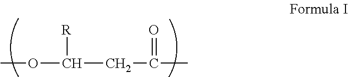

Preferably, the polymer is polyhydroxyalkanoate. The polymer can be a copolymer including poly(3-hydroxybutyrate-co-3-hydroxyvalerate) random copolymer (P(3HB-co-3HV)) or poly(3-hydroxybutyrate-co-3-hydroxyhexanoate) random copolymer [P(3HB-co-3HHx)]. The transformant carrying the polymer synthase gene has the ability to produce P(3HB-co-3HHx) with very high efficiency.

The convention process for producing poly(3-hydroxybutyrate) [P(3HB)] causes problem in physical properties of inferior resistance to impact because this polymer is a highly crystalline polymer. Degree of crystallinity is lowered by introducing 3-hydroxyvalerate having 5 carbon atoms or 3-hydroxyhexanoate having 6 carbon atoms into a polymer chain. The polymer acts as a flexible polymeric material which is also excellent in thermostability and formability.

In the present invention, the P(3HB-co-3HHx) copolymer can be produced in high yield by use of the polymer synthase of Chromobacterium sp. used. Since the desired polymer can be obtained in a large amount using the above means, it can be used as a biodegradable material of yarn, film or various vessels. Further, the gene of the present invention can be used to breed a strain highly producing the P(3HB-co-3HHx) copolymer.

The present disclosure includes as contained in the appended claims, as well as that of the foregoing description. Although this invention has been described in its preferred form with a degree of particularity, it is understood that the present disclosure of the preferred form has been made only by way of example and that numerous changes in the details of construction and the combination and arrangements of parts may be resorted to without departing from the scope of the invention.

### EXAMPLE

Examples are provided below to illustrate different aspects and embodiments of the present invention. These examples are not intended in any way to limit the disclosed invention, which is limited only by the claims.

### Example 1

### Cloning of the Polymer Synthase Gene from Chromobacterium sp. USM2

Initially, genomic DNA library was isolated from Chromobacterium sp. USM2. Chromobacterium sp. USM2 was cultured overnight in 50 ml nutrient rich medium (1% peptone, 1% meat extract, 0.5% yeast extract, pH 7.0) at 30° C. and then genomic DNA was obtained from the microorganism using the standard method.

To obtain a DNA fragment containing the polymer synthase gene from Chromobacterium sp. USM2, a probe was then prepared. Two domain-specific oligonucleotides designed using NCBI database as a reference, SEQ ID NO:3 and SEQ ID NO:4, were synthesized.

The polymer synthase gene was amplified by PCR using these oligonucleotides as primers and the genomic DNA from Chromobacterium sp. USM2 as a template. PCR was carried out using 30 cycles, each consisting of reaction at 95° C. for 20 seconds, 60° C. for 180 seconds, and 60° C. for 180 seconds.

The nucleotide sequence of a 1.7 kbp ApaI-SalI from this fragment was determined by the Sanger method. The polymer synthase gene containing the nucleotide sequence (1704) SEQ ID NO:1 was obtained.

### Example 2

### Preparation of Cuprividus necator Transformant

The ApaI-SalI polymer synthase gene fragment was first inserted into a cloning vector pGEM-T (Promega) previously cleaved with the same restriction enzyme. The fragment was then digested again with ApaI and SalI restriction enzymes and the resulting ApaI-SalI polymer synthase gene fragment was inserted into a recombinant vector pBBR1MCS-2 capable of expression in microorganisms belonging to the genus Cupriavidus, and the resulting recombinant plasmid was transformed into Cupriavidus necator PHB-4 (DSM 541) (strain deficient in the ability to synthesize polymer) by the conjugation transfer method.

Firstly, the recombinant plasmid was used to transform E. coli S17-1 by the calcium chloride method. The recombinant E. coli thus obtained and C. necator PHB-4 were transconjugated. The recombinant E. coli and C. necator PHB-4 were cultured overnight in 1.5 ml LB medium and nutrient rich medium at 30° C., and the respective cultures, each 0.1 ml, were combined and cultured on a shaker at room temperature for 1 hour. The mixture was then incubated without shaking for 30 minutes, and subsequently shaken again for 30 minutes. This microbial mixture was plated on Simmon's citrate agar containing 50 mg/L kanamycin and cultured at 30° C. for 2 days.

Since C. necator PHB-4 is rendered resistant to kanamycin by transferring the plasmid in the recombinant E. coli into it, the colonies grown on the Simmon's citrate agar are a transformant of C. necator.

### Example 3

### Synthesis of Polymer by C. necator Transformants

Each of C. necator 1116, C. necator transformant and PHB-4 were inoculated into 50 ml mineral medium (3.32 g/L disodium hydrogen phosphate, 2.8 g/L potassium dihydrogen phosphate, 0.54 g/L urea) containing 1 ml/L of trace elements and incubated in a flask at 30° C. 50 mg/L kanamycin was added in the mediums for C. necator transformants and the microorganisms were cultured for 48 and 72 hours.

Each of strains H16, C. necator transformant and P11B-4 was inoculated into the above mineral medium to which 5 g/L fructose and crude palm kernel oil (CPKO) had been added, and each strain was cultured at 30° C. for 72 hours in a 250 ml flask. Sodium valerate (2.5 g/L) was added for 3-hydroxyvalerate (3HV) generation. 50 mg/L kanamycin was added in the mediums for C. necator transformants. For poly(3- hydroxybutyrate-co-3-hydroxyvalerate-co-3-hydroxyhexanoate) [P(3HB-co-3HV-co-3HHx)] terpolymer synthesis, various concentrations of sodium valerate and sodium propionate was added together with 12 g/L of CPKO.

The microorganisms were recovered by centrifugation, washed with distilled water and hexane (in the presence of CPKO) and lyophilized, and the weight of the dried microorganisms was determined. 2 ml sulfuric acid/methanol mixture (15:85) and 2 ml chloroform were added to 10-30 mg of the dried microorganism, and the sample was sealed and heated at 100° C. for 140 minutes whereby the polymer in the microorganisms was decomposed into methylester. 1 ml distilled water was added thereto and stirred vigorously. It was left and separated into 2 layers, and the lower organic layer was removed and analyzed for its components by capillary gas chromatography through a capillary column Neutra BOND-1 (column of 25 m in length, 0.25 mm in inner diameter and 0.4 μm in liquid film thickness, manufactured by GL Science) in Shimadzu GC-2010. The temperature was raised at a rate of 8° C./min. from an initial temperature of 100° C. The results are shown in Tables 1, 2 and 3.

Table 1 shows the biosynthesis of PHA by C. necator transformant from fructose, mixture of fructose and sodium valerate and CPKO.

TABLE 1

| | | Cell dry | PHA | PHA composition | | |
|---|---|---|---|---|---|---|
| Carbon | | weight | content | (mol %) | | |
| source | Strain | (g/L) | (wt %)^{b} | 3HB | 3HV | 3HHx |
| Fructose | Transfor- | 3.1 ± 0.2 | 64 ± 2 | 100 | ND | ND |
| | mant | | | | | |
| | H16 | 3.3 ± 0.1 | 56 ± 1 | 100 | ND | ND |
| | PHB-4 | 2.3 ± 0.2 | 0 | ND | ND | ND |
| Fructose + | Transfor- | 2.8 ± 0.2 | 57 ± 2 | 40 | 60 | ND |
| Sodium | mant | | | | | |
| valerate | H16 | 3.0 ± 0.1 | 38 ± 2 | 65 | 35 | ND |
| CPKO | Transfor- | 4.0 ± 0.2 | 63 ± 2 | 96 | ND | 4 |
| | mant | | | | | |
| | H16 | 5.1 ± 0.4 | 60 ± 1 | 100 | ND | ND |

^{a}Incubated for 48 hours at 30.degree. C., initial pH 7.0, 200 rpm in mineral medium.
Sodium valerate was added at 24 hours of cultivation.
^{b}PHA content in freeze-dried cells
3HB, 3-hydroxybutyrate; 3HV, 3-hydroxyvalerate; 3HHx, 3-hydroxyhexanoate
ND—Not detected

Based on the results in Table 1, the transformant could utilize fructose for the production of P(3HB) homopolymer. Cell dry weight of 3.1±0.2 g/L and polymer content of 64±2% by weight of the microorganism was almost similar to that of H16 (3.3±0.1 g/L and 56±1% by weight of the microorganism). As expected, no accumulation was observed in PHB-4. Higher cell dry weight was obtained when CPKO was used as the sole carbon source. The cell biomass of the transformant was 4.0±0.2 g/L and the polymer content was 63±2% by weight of the microorganism.

Interestingly, in the presence of CPKO, accumulation of P(3HB-co-3HHx) copolymer with 4 mol % of 3HHx was observed in the transformant. This was not evident in the wild type Chromobacterium sp. USM2 or H16.

To investigate the production of P(3HB-co-3HV) copolymer, sodium valerate was added to the culture supplemented with fructose. The 3HV composition generated by the transformant was nearly 2-fold higher compared to H16. Polymer content produced by this recombinant was also higher at 57±2% by weight of the microorganism compared to the wild type, 38±2% by weight of the microorganism. The ability of the cloned polymer synthase to accumulate high amount of 3HV from lower concentration of precursor indicates its high affinity towards the incorporation of 3HV.

Table 2 shows the time profile analysis of P(3HB-co-3HHx) accumulation by C. necator transformant from CPKO.

TABLE 2

| | | | |
|---|---|---|---|
| | Cell dry | PHA | PHA composition |
| Time | weight | content | (mol %) |

| (h) | (g/L) | (wt %)^{b} | 3HB | 3HHx |
|---|---|---|---|---|
| 6 | 0.4 | 5 ± 1 | 89 | 11 |
| 12 | 1.8 ± 0.1 | 27 ± 4 | 91 | 9 |
| 24 | 4.5 ± 0.6 | 45 ± 2 | 95 | 5 |
| 36 | 5.5 ± 0.6 | 65 ± 1 | 96 | 4 |
| 48 | 7.1 ± 0.4 | 76 ± 2 | 97 | 3 |
| 60 | 8.2 ± 0.7 | 81 ± 2 | 97 | 3 |
| 72 | 8.8 ± 0.5 | 83 ± 4 | 97 | 3 |

PHA, polyhydroxyalkanoate; P(3HB), poly(3-hydroxybutyrate); P(3HHx), poly(3-hydroxyhexanoate)
^{a}Incubated for 72 hours at 30.degree. C., initial pH 7.0, 200 rpm in PHA biosynthesis medium
CPKO was added during inoculation (0 h)
^{b}PHA content in freeze-dried cells were determined via gas chromatography (GC)

From the results in Table 2, the 3HHx mol % fraction could be controlled based on the duration of cultivation and a range from 3 to 11 mol % could be produced. High cell biomass and polymer content was obtained when cultured for 72 hours. Total cell dry weight of 8.8±0.5 g/L and polymer content of 83±4% by weight of the microorganism was obtained. Cells were predicted to be utilizing the supplied carbon source efficiently, as indicated by the substantial decrease in the concentration of residual oil until none was detected at the end of 72 hours cultivation.

Table 3 shows the biosynthesis of P(3HB-co-3HV-co-3HHx) terpolymer by C. necator transformant from mixtures of CPKO and various concentrations of precursors.

TABLE 3

| | | | |
|---|---|---|---|
| | Cell dry | PHA | PHA composition |
| Precursor | weight | content^{b} | (mol %) |

| | | | | | |
|---|---|---|---|---|---|
| (g/L) | (g/L) | (wt %) | 3HB | 3HV | 3HHx |

Sodium valerate

| | | | | | |
|---|---|---|---|---|---|
| 1 | 6.9 ± 2.0 | 10 ± 1 | 69 | 24 | 7 |
| 3 | 1.9 ± 0.2 | 23 ± 6 | 19 | 79 | 2 |
| 5 | 2.2 ± 0.1 | 33 ± 6 | 10 | 89 | 1 |
| 7 | 3.5 ± 0.1 | 53 ± 4 | 14 | 85 | 1 |
| 9 | 2.1 ± 0.4 | 69 ± 1 | 8 | 91 | 1 |

Sodium propionate

| | | | | | |
|---|---|---|---|---|---|
| 1 | 6.0 ± 0.2 | 32 ± 1 | 91 | 1 | 8 |
| 3 | 1.6 ± 0.1 | 2 ± 1 | 71 | 23 | 6 |
| 5 | 2.2 ± 0.8 | 10 ± 4 | 47 | 49 | 4 |
| 7 | 1.9 ± 0.1 | 11 ± 3 | 30 | 69 | 1 |
| 9 | 1.8 ± 0.3 | 35 ± 3 | 48 | 51 | 1 |

^{a}Incubated for 72 hours at 30.degree. C., initial pH 7.0, 200 rpm in PHA biosynthesis medium. Precursors were added at 6 hours of cultivation.
CPKO was added during inoculation (0 hour)
^{b}PHA content in freeze-dried cells were determined via gas chromatography (GC)

As shown in Table 3, the 3HV mol % fraction could be regulated by adding a range of different precursor concentration. Generally, it was found that the 3HV mol % increased with respect to increasing concentration of precursor. With sodium valerate, the 3HV mol % ranged from 24 to 91 mol %. Subsequently, with sodium propionate it ranged from 1 to 51 mol %.

The 3HHx mol % fraction was higher when lower concentrations of precursors were used. It was 7 mol % and 8 mol % with 1 g/L sodium valerate and sodium propionate respectively. Highest polymer content of 69±1 by weight of the microorganism was produced when 9 g/L of sodium valerate was used. Generally, cell biomass was higher when a lower concentration of these precursors was fed.

Thereafter, the dried C. necator transformant cells are suspended in chloroform and heated to 60° C. for 4 hours to extract polymer from it. The residues are removed by filtration. Methanol is added to this chloroform solution to precipitate polymer. After the supernatant is removed by filtration or centrifugation, the precipitates are dried to give purified polymer.

The resulting polymer is confirmed by nuclear magnetic resonance. A total of 25 mg of polymer sample is dissolved in 1 ml of deuterated chloroform (CDCl₃). The ¹H NMR spectra were measured on a Bruker AVANCE 300; NC, USA spectrometer at 400 MHz at 30 ° C. Tetramethylsilane (Me₄Si) was used as an internal chemical shift reference. The result is shown in FIG. 4.

1. An isolated polynucleotide encoding for a polypeptide comprising an amino acid sequence set forth in SEQ ID NO: 1 with polymer synthase activity.

2. An isolated polynucleotide according to claim 1 comprising a nucleotide sequence set forth in SEQ ID NO: 2 or the complementary sequence thereof.

3. An isolated polynucleotide according to claim 1 comprising a nucleotide sequence set forth in SEQ ID NO: 2, wherein T is replaced by U; or the complementary sequence thereof.

4. A recombinant vector comprising an isolated polynucleotide according to claim 1.

5. A recombinant vector according to claim 4 which is a plasmid or phage.

6. A transformant transformed by the vector according to claim 5.

7. A process for producing polymer comprising:
culturing a transformant according to claim 6 in a medium containing polymerizable materials; and
recovering the polymer from the cultured medium.

8. A process according to claim 7, wherein the polymer is polyhydroxyalkanoate.

9. An isolated polynucleotide encoding for a polypeptide comprising an amino acid sequence set forth in SEQ ID NO: 1, wherein one or more amino acids is replaced, deleted, replaced or added, the polypeptide having polymer synthase activity.

10. A recombinant vector comprising an isolated polynucleotide according to claim 9.

11. A recombinant vector according to claim 10 which is a plasmid or phage.

12. A transformant transformed by the vector according to claim 11.

13. A process for producing polymer comprising:
culturing a transformant according to claim 12 in a medium containing polymerizable materials; and
recovering the polymer from the cultured medium.

14. A process according to claim 13, wherein the polymer is polyhydroxyalkanoate.


SEQUENCE LISTING (4 sequences)

SEQ ID NO: 1 - protein, 567 aa, Artificial
  Synthetic Construct
MQQFVNSLSLGQDQSDAPHPLTGAWSQLMSQTNQLLQLQSSLYQQQLGLWTQFLGQTAGNDASAPSAKPSDRRFASPEWDEHPFYSFLKQSYLQTSKWMMELVDKTQIDESAKDKLSFATRQYLDAMAPSNFMLTNPDVVKRAIETQGESLVEGMKNMMEDIQKGHISMSDESKFQIGKNLVVTPGEVVFRNELIELIQYTPTTEKVHEKPLLFVPPCINKYYLMDLQPDNSMVRHFVGQGYRVFLVSWRSAVPEMKNFTWETYIEKGVFAAAEAVQKITKQPTMNALGFCVGGVILTTALCVAQAKGLKYFDSATFMTSLIDHAEPGEISFFIDEALVASREAKMAAGGIISGKEIGRTFASLRANDLVWNYVVNNYLLGKTPAPFDLLYWNNDAVDLPLPMHTFMLRQFYINNALITPGAITLCGVPIDISKIDIPVYMFAAREDHIVLWSSAYSGLKYLSGTPSRRFVLGASGHIAGSINPVTKDKRNYWTNEQLPVNPEEWLEGAQSHPGSWWKDWDAWLAPQSGKQVPAPKMLGSKEFPPLQPAPGSYVLAKAMPPVAAALN

SEQ ID NO: 2 - DNA, 1704 bp, Artificial
  Synthetic Construct
atgcagcagtttgtcaattccctgtcgctcggccaggaccagtccgatgccccccatccgctgaccggcgcctggtcgcagctgatgagccagaccaatcagctcttgcagctgcaatccagcctctaccaacagcaattgggcttgtggacgcaattcctcggccaaaccgccggcaacgacgcttccgcgccgtcggccaagccgagcgaccgccgcttcgcctcgccggagtgggatgagcatccgttctacagcttcctcaagcagagctatctgcaaacctccaagtggatgatggagctggtggacaagacccagatcgacgaaagcgctaaggacaagctgtccttcgccactcgccagtacctggatgccatggcgcccagcaacttcatgctgaccaaccccgacgtggtcaaacgcgccatcgaaacccagggcgaaagcctggtggaaggcatgaagaacatgatggaggacatccagaagggccatatctcgatgtccgacgagagcaagttccaaatcggcaaaaacctggtggtgacgccgggcgaggtggtgttccgcaatgaactgatcgagctgatccagtacacgccgaccaccgaaaaagtccacgaaaagcccttgctgtttgtcccgccgtgcatcaacaagtactacctgatggatctgcagccggacaactccatggtgcgccacttcgtcggccagggttaccgcgttttcctggtcagctggcgttccgccgtgcccgagatgaagaacttcacttgggagacctacatcgagaaaggcgtgttcgccgcggccgaagcggttcagaagatcaccaagcagccgaccatgaacgcgctgggcttctgcgtgggcggcgtgatcctcactaccgcgctgtgcgtggcccaggccaagggtctgaaatacttcgactccgccaccttcatgacgtcgctgatcgaccacgccgaaccgggcgagatctccttcttcatcgacgaggcgctggtagccagccgcgaagccaaaatggcggccggcggcatcatcagcggcaaggaaatcggacgcactttcgccagcctgcgcgccaacgacctggtgtggaactacgtcgtcaacaactacctgctgggcaagaccccggcgccgttcgacctgctgtactggaacaacgacgcggtggacctgccgctgccgatgcacaccttcatgctgcggcagttctacatcaacaacgcgctgatcaccccgggcgccattacgctgtgcggcgtcccgatcgacatctccaagatcgacatcccggtgtatatgttcgccgcgcgcgaagaccacatcgtgctgtggagttccgcctactccggcctgaaatatctgagcggcacgccaagccgccgctttgtcctgggcgcatccggccacatcgccggctcgatcaaccccgtcaccaaggataagcgcaactactggaccaacgagcagctgccggtgaatccggaagaatggctggagggcgcgcaaagccatccaggcagctggtggaaggactgggacgcctggctggccccgcaatctggaaaacaggttccggcgcccaagatgctgggcagcaaggagttccccccgctgcagcctgcgccgggcagctatgtgctcgccaaggcgatgcctcccgtcgccgccgccttgaactga

SEQ ID NO: 3 - DNA, 20 bp, Artificial
  Synthetic Construct
cgtaattggggcccatgcag

SEQ ID NO: 4 - DNA, 25 bp, Artificial
  Synthetic Construct
agccgccgccgaagcttccgatggc